(19) United States
(12) Patent Application Publication
(10) Pub. No.: US 2005/0222441 A1
(43) Pub. Date: Oct. 6, 2005

(54) Process for preparing a catalyst, the catalyst, and a use of the catalyst

(76) Inventor: Jian Lu, Houston, TX (US)

Correspondence Address: SHELL OIL COMPANY, P O BOX 2463, HOUSTON, TX, 772522463, US

(21) Appl. No.: 11/095,336

(22) Filed: Mar. 31, 2005

Related U.S. Application Data
(60) Provisional application No. 60/558,464, filed on Apr. 1, 2004.

(51) Int. Cl. C07D 301/03; C07C 43/03

(52) U.S. Cl. 549/534; 564/471; 568/679; 568/867

(57) ABSTRACT

A process for preparing a catalyst comprising silver on a support, which process comprises depositing silver on the support, and
depositing on the support a base having a pK_{b} of at most 3.5 when measured in water at 25° C., in a quantity of at least 50 mmole per kg of the support, simultaneously with or later than depositing silver on the support; the catalyst; and a process for preparing an olefin oxide by reacting an olefin with oxygen in the presence of the catalyst.

### FIELD OF THE INVENTION

The invention relates to a process for preparing a catalyst suitable for the epoxidation of an olefin, the catalyst per se and a process for the epoxidation of an olefin in which the catalyst is used.

### BACKGROUND OF THE INVENTION

In olefin epoxidation an olefin is reacted with oxygen to form an olefin epoxide, using a silver-based catalyst. The olefin oxide may be reacted with water, an alcohol or an amine to form a 1,2-diol, a 1,2-diol ether or an alkanolamine. Thus, 1,2-diols, 1,2-diol ethers and alkanolamines may be produced in a multi-step process comprising olefin epoxidation and converting the formed olefin oxide with water, an alcohol or an amine.

Conventional silver-based catalysts have provided the olefin oxide notoriously in a low selectivity. Also, the catalysts are subject to an aging-related performance decline during normal operation. The aging manifests itself by a reduction in the activity of the catalyst. Usually, when a reduction in activity of the catalyst is manifest, the reaction temperature is increased in order to compensate for the reduction in activity. The reaction temperature may be increased until it becomes undesirably high, at which point in time the catalyst is deemed to be at the end of its lifetime and would need to be exchanged.

Over the years much effort has been devoted to improving epoxidation catalysts in their performance, for example in respect of their initial activity and selectivity, and in respect of their stability performance, that is their resistance against the aging-related performance decline. Solutions have been found in improved compositions of the catalysts, and, in other instances, solutions have been found in improved processes of preparing the catalysts.

For example, the silver-based catalysts are customarily made by depositing silver on the support by using an impregnation technique. U.S. Pat. No. 6,368,998 shows that the use of a silver containing impregnation solution which has a higher pH than conventional, for example having a measured pH of 13.2 or 13.6, by the presence therein of additional base in the form of hydroxide, leads to catalysts which have improved initial performance properties. U.S. Pat. No. 6,368,998 teaches that high additions of hydroxides have been seen to cause sludging of the impregnation solution, creating manufacturing difficulties. In U.S. Pat. No. 6,368,998 there is no teaching relating to the catalysts' stability performance.

### SUMMARY OF THE INVENTION

In a first embodiment, amongst others, the present invention provides a process for preparing a catalyst comprising silver on a support, which process comprises
depositing silver on the support, and
depositing on the support a base having a pK_{b} of at most 3.5 when measured in water at 25° C., in a quantity of at least 50 mmole per kg of the support, simultaneously with or later than depositing silver on the support.

In the first embodiment, the invention preferably provides a process for preparing the catalyst which process comprises impregnating the support with a solution comprising a silver compound and a base having a pK_{b} of at most 3.5 when measured in water at 25° C. in a quantity of at least 50 mmole per kg of the support. In a second embodiment, the present invention provides a process for preparing a catalyst comprising silver on a support, which process comprises
depositing silver on the support, and
depositing on the support a component comprising lithium in a quantity comprising at least 50 mmole of lithium per kg of the support, simultaneously with or later than depositing silver on the support.

In the second embodiment, the invention preferably provides a process for preparing the catalyst which process comprises impregnating the support with a solution comprising a silver compound and a component comprising lithium in a quantity of at least 50 mmole of lithium per kg of the support.

The present invention also provides a catalyst obtainable by the process for preparing a catalyst in accordance with this invention.

The present invention also provides a process for preparing an olefin oxide by reacting an olefin with oxygen in the presence of a catalyst obtainable by the process for preparing a catalyst in accordance with this invention.

The present invention also provides a method of using an olefin oxide for making a 1,2-alkanediol, a 1,2-alkanediol ether or a 1,2-alkanolamine comprising converting the olefin oxide into the 1,2-alkanediol, the 1,2-alkanediol ether, or the 1,2-alkanolamine, wherein the olefin oxide has been obtained by a process for preparing an olefin oxide according to this invention.

### DETAILED DESCRIPTION OF THE INVENTION

When a catalyst is prepared in accordance with this invention the performance decline of the catalyst during its use is fundamentally changed, compared with a conventionally prepared catalyst. The fundamental change is advantageous because it resides in a postponement over a substantial period of time of the normally seen relatively rapid decline in selectivity of the catalyst. During the period of postponement, there is virtually no selectivity decline or the selectivity decline is at a substantially lower rate. Further, the catalysts of this invention exhibit an advantage in their initial performance, in particular in their initial activity.

The support for use in this invention may be based on a wide range of materials. Such materials may be natural or artificial inorganic materials and they may include refractory materials, silicon carbide, clays, zeolites, charcoal and alkaline earth metal carbonates, for example calcium carbonate. Preferred are refractory materials, such as alumina, magnesia, zirconia and silica. The most preferred material is α-alumina. Typically, the support comprises at least 85 % w, more typically 90 % w, in particular 95 % w α-alumina, frequently up to 99.9 % w α-alumina, relative to the weight of the support. Other components of the α-alumina support may comprise, for example, silica, alkali metal components, for example sodium and/or potassium components, and/or alkaline earth metal components, for example calcium and/or magnesium components.

The surface area of the support may suitably be at least 0.1 m²/g, preferably at least 0.3 m²/g, more preferably at least 0.5 m²/g, and in particular at least 0.6 m²/g, relative to the weight of the support; and the surface area may suitably be at most 10 m²/g, preferably at most 5 m²/g, and in particular at most 3 m²/g, relative to the weight of the support. “Surface area” as used herein is understood to relate to the surface area as determined by the B.E.T. (Brunauer, Emmett and Teller) method as described in the Journal of the American Chemical Society 60 (1938) pp. 309-316. High surface area supports, in particular when they are α-alumina supports optionally comprising in addition silica, alkali metal and/or alkaline earth metal components, provide improved performance and stability of operation.

The water absorption of the support is typically in the range of from 0.2 to 0.8 g/g, preferably in the range of from 0.3 to 0.7 g/g. A higher water absorption may be favored in view of a more efficient deposition of silver and further elements, if any, on the support by impregnation. However, at a higher water absorption, the support, or the catalyst made therefrom, may have lower crush strength. As used herein, water absorption is deemed to have been measured in accordance with ASTM C20, and water absorption is expressed as the weight of the water that can be absorbed into the pores of the support, relative to the weight of the support.

The support is typically a calcined, i.e. sintered, support, preferably in the form of formed bodies, the size of which is in general determined by the dimensions of a reactor in which they are to be deposited. Generally however it is found very convenient to use particles such as formed bodies in the form of powdery particles, trapezoidal bodies, cylinders, saddles, spheres, doughnuts, and the like. The cylinders may be solid or hollow, straight or bent, and they may have their length and cross-sectional dimensions about the same and from 5 to 15 mm.

The performance of the catalyst may be enhanced if the support is washed before depositing catalyst ingredients on the support. On the other hand, unwashed supports may also be used successfully. A useful method for washing the support comprises washing the support in a continuous fashion with hot, demineralized water, until the electrical conductivity of the effluent water does not further decrease. A suitable temperature of the demineralized water is in the range of 80 to 100° C., for example 90 ° C or 95° C. Alternatively, the support may be washed with base and subsequently with water. Reference may be made to U.S. Pat. No. 6,368,998, which is incorporated herein by reference.

The washing is intended to remove soluble residues from the support, in particular soluble residues which can be measured as nitric acid extractable components of the support. A method of measuring the content of nitric acid extractable components involves extracting a 10-gram sample of the support by boiling it with a 100 ml portion of 10% w nitric acid for 30 minutes (1 atm., i.e. 101.3 kPa) and determining in the combined extracts the relevant components by using a known method, for example atomic absorption spectroscopy. Reference is made to U.S. Pat. No. 5,801,259, which is incorporated herein by reference. The support for use in this invention, or more generally a support for preparing silver-based catalysts for use in the preparation of an olefin oxide from the olefin and oxygen, has typically a content of nitric acid extractable components (as the weight of the metal, or SiO₂), relative to the weight of the support; in parts per million (ppmw) as follows:
sodium: less than 500 ppmw, preferably less than 400 ppmw, and/or
potassium: less than 150 ppmw, preferably less than 100 ppmw, and/or
calcium: less than 400 ppmw, preferably less than 300 ppmw, and/or
aluminum: less than 1100 ppmw, preferably less than 800 ppmw, and/or
silicate: less than 1000 ppmw, preferably less than 800 ppmw.

If the support is a calcined support, in particular an α-alumina support, re-calcining the support may be an alternative method of reducing the content of nitric acid extractable components of the support. Suitably, the calcination is carried out by heating a precursor of the support at a temperature in the range of from 1000 to 1600° C., preferably 1200 to 1500° C., typically for a period of from 1 to 50 hours, and more typically from 10 to 40 hours. Suitably, the re-calcination may be carried out by heating the support at a similar temperature and for a similar period of time as in the calcination. Preferably, the conditions of re-calcination are somewhat less severe than the conditions of calcination, for example in that the temperature is 50° C. or 100° C. lower and/or the time is shorter. The atmosphere applied in the calcination or re-calcination is not critical. For example, an inert atmosphere may be applied, such as nitrogen or argon, or an oxygen-containing atmosphere may be applied such as air or a mixture of air and nitrogen.

In a preferred embodiment, amongst others, a base (hereinafter “first base”) may be deposited on the support before depositing catalyst ingredients on the support. Suitably, the first base has a pK_{b} of at most 3.5, when measured in water at 25° C., preferably, the pK_{b} is at most 2, more preferably at most 1. A suitable first base may be a hydroxide, for example lithium hydroxide or a quaternary ammonium hydroxide, typically tetramethylammonium hydroxide or tetraethylammonium hydroxide, or an alkoxide, typically lithium methoxide or aluminum trimethoxide. The quantity of the first base may be up to 1000 mmole/kg support, for example in the range of from 0.5 to 500 mmole/kg support, preferably in the range of from 1 to 100 mmole/kg, more preferably in the range of from 5 to 50 mmole/kg, for example 10, 14, 20 or 30 mmole/kg.

The skilled person will appreciate that a base may be multibasic, that is having a multitude of basic functionalities. For example, a base may be dibasic or tribasic. The base properties of a multibasic compound may be specified using more than one pK_{b} value. It is to be understood that, as used herein, in the case of a multibasic compound the number of moles of base having a pK_{b} as specified includes the total number of moles of basic functionalities having the specified pK_{b} value.

The first base may be deposited on the support by impregnating the support with a solution containing a sufficient amount of the first base. After impregnation, the support may be dried, typically at a temperature of at most 300° C., preferably at most 250° C., more preferably at most 200° C., and suitably at a temperature of at least 20 ° C., preferably at least 50° C., more preferably at least 80 ° C., suitably for a period of time of at least 1 minute, preferably at least 2 minutes, and suitably for a period of time of at most 60 minutes, preferably at most 30 minutes, more preferably at most 15 minutes. The application of more severe conditions, up to the calcination conditions, as described hereinbefore, may be considered in addition to, or in place of, the conditions described for the drying.

The volume of impregnation solutions described herein may be such that the support is impregnated until a point of incipient wetness of the support has been reached. Alternatively, a larger volume may be used and the surplus of solution may be removed from the wet support, for example by decantation or centrifugation. Amongst others, the impregnation solutions may comprise an alcoholic diluent, for example methanol of ethanol, or it may be aqueous. This includes that mixed diluents may be used.

The preparation of silver-based catalysts is known in the art and the known methods are applicable to the preparation of the catalyst in accordance with the invention. Methods of preparing the catalyst include impregnating the support with a silver compound containing cationic silver and performing a reduction to form metallic silver particles. Reference may be made, for example, to U.S. Pat. No. 5,380,697, U.S. Pat. No. 5,739,075, EP-A-266015, and U.S. Pat. No. 6,368,998, which U.S. patents are incorporated herein by reference.

The reduction of cationic silver to metallic silver may be accomplished during a step in which the catalyst is dried, so that the reduction as such does not require a separate process step. This may be the case if the silver containing impregnation solution comprises a reducing agent, for example, an oxalate, as described in the Examples hereinafter. Such drying step is suitably carried out at a reaction temperature of at most 300° C., preferably at most 280° C., more preferably at most 260° C., and suitable at a reaction temperature of at least 200° C., preferably at least 210° C., more preferably at least 220° C., suitably for a period of time of at least 1 minute, preferably at least 2 minutes, and suitably for a period of time of at most 20 minutes, preferably at most 15 minutes, more preferably at most 10 minutes.

Appreciable catalytic activity is obtained by employing a silver content of the catalyst of at least 10 g/kg, relative to the weight of the catalyst. Preferably, the catalyst comprises silver in a quantity of from 50 to 500 g/kg, more preferably from 100 to 400 g/kg, for example 105 g/kg, or 120 g/kg, or 190 g/kg, or 250 g/kg, or 350 g/kg, relative to the weight of the catalyst. The silver compound may be employed in the impregnation solution in a quantity sufficient to provide in a single deposition of silver a catalyst having a content of silver as disclosed herein. Alternatively, multiple depositions of silver may be applied.

In accordance with an embodiment of this invention, a base (hereinafter “second base”) may be deposited on the support simultaneously with or later than depositing silver on the support. The second base has a pK_{b} of at most 3.5, when measured in water at 25° C., preferably, the pK_{b} is at most 2, more preferably at most 1. A suitable second base may be a hydroxide, for example lithium hydroxide or a quaternary ammonium hydroxide, typically tetramethylammonium hydroxide or tetraethylammonium hydroxide, or an alkoxide, typically lithium methoxide or aluminum trimethoxide. The quantity of second base is at least 50 mmole/kg support, preferably at least 55 mmole/kg, more preferably at least 60 mmole/kg. The quantity of base is typically at most 1000 mmole/kg support, preferably at most 500 mmole/kg, more preferably at most 100 mmole/kg, for example 70 or 75 mmole/kg support.

The second base is preferably deposited on the support simultaneously with depositing silver on the support, in which case the second base may be added to a silver containing impregnation solution, described hereinbefore. Preferably, the pH of the impregnation solution is at least 14, in particular at least 14.5. Alternatively, the second base may be deposited on the support after depositing silver, typically by applying a separate impregnation. In the separate impregnation, the second base may be deposited on the support, optionally together with other catalyst ingredients. As used herein, “pH” is the pH as measured at 20° C. The measured pH may be different from the true pH, because the medium of the solution of which the pH is measured may not be aqueous.

The second base may or may not be the same as the first base. The second base may be deposited on the support independent of whether or not the first base is deposited on the support. The first base may be deposited on the support independent of whether or not the second base is deposited on the support.

In accordance with another embodiment of this invention, a compound comprising lithium may be deposited on the support simultaneously with or later than depositing silver on the support. The compound comprising lithium may be, for example, lithium hydroxide or a lithium salt, for example lithium nitrate or lithium sulfate. The quantity of the compound comprising lithium is at least 50 mmole/kg support, preferably at least 55 mmole/kg, more preferably at least 60 mmole/kg. The quantity of the compound comprising lithium is typically at most 1000 mmole/kg support, preferably at most 500 mmole/kg, more preferably at most 100 mmole/kg, for example 75 mmole/kg support.

The compound comprising lithium is preferably deposited on the support simultaneously with depositing silver on the support, in which case the compound comprising lithium may be added to a silver containing impregnation solution, described hereinbefore. Alternatively, the compound comprising lithium may be deposited on the support after depositing silver, typically by applying a separate impregnation. In the separate impregnation, the compound comprising lithium may be deposited on the support, optionally together with other catalyst ingredients.

Preferably, the catalyst comprises, in addition to silver, one or more high-selectivity dopants. Catalysts comprising a high-selectivity dopant are known from U.S. Pat. No. 4,761,394 and U.S. Pat. No. 4,766,105, which are incorporated herein by reference. The high-selectivity dopants may comprise, for example, components comprising one or more of rhenium, molybdenum, chromium and tungsten. The high-selectivity dopants may be present in a total quantity of from 0.01 to 500 mmole/kg, calculated as the element (for example, rhenium, molybdenum, tungsten, and/or chromium) on the total catalyst. Rhenium, molybdenum, chromium or tungsten may suitably be provided as an oxide or as an oxyanion, for example, as a perrhenate, molybdate, tungstate, in salt or acid form. The high-selectivity dopants may be employed in the invention in a quantity sufficient to provide a catalyst having a content of high-selectivity dopant as disclosed herein.

Of special preference are catalysts which comprise a rhenium component, and optionally a rhenium co-promoter, in addition to silver. The rhenium component may typically be present in a quantity of at least 0.01 mmole/kg, more typically at least 0.1 mmole/kg, and preferably at least 0.5 mmole/kg, calculated as the quantity of rhenium relative to the weight of the catalyst. The rhenium component may be present in a quantity of at most 50 mmole/kg, preferably at most 10 mmole/kg, more preferably at most 5 mmole/kg, calculated as the quantity of rhenium relative to the weight of the catalyst. The rhenium co-promoter may suitably be selected from components which comprise one or more of tungsten, chromium, molybdenum, sulfur, phosphorus and boron. Preferably, the rhenium copomoter is selected from components which comprise one or more of tungsten, chromium, molybdenum and sulfur. It is particularly preferred that the rhenium co-promoter comprises a tungsten component. The rhenium co-promoter may typically be present in a total quantity of at least 0.01 mmole/kg, more typically at least 0.1 mmole/kg, and preferably at least 0.5 mmole/kg, calculated as the element (i.e. the total of tungsten, chromium, molybdenum, sulfur, phosphorus and/or boron), relative to the weight of the catalyst. The rhenium co-promoter may be present in a total quantity of at most 50 mmole/kg, preferably at most 10 mmole/kg, more preferably at most 5 mmole/kg, on the same basis. The form in which the rhenium co-promoter may be deposited is not material to the invention. For example, it may suitably be provided as an oxide or as an oxyanion, for example, as a sulfate, borate or molybdate, in salt or acid form. The rhenium component and the rhenium co-promoter may be employed in the invention in quantities sufficient to provide a catalyst having contents of the rhenium component and rhenium co-promoter as disclosed herein.

The high-selectivity dopants, if any, may or may not be deposited on the support together with the deposition of silver. In the preparation of a catalyst having a relatively high silver content, for example in the range of from 150 to 500 g/kg, in particular from 200 to 400 g/kg, on total catalyst, it may be advantageous to apply multiple depositions of silver. Silver may be deposited in three or more portions, and preferably in two portions, which depositions may be together with or separate from the deposition of high-selectivity dopants. If a rhenium component and a rhenium co-promoter are present, a portion of silver may be deposited together with the deposition of the rhenium co-promoter, and another portion may be deposited together with the deposition of the rhenium component. Without wishing to be bound by theory, it is believed that in embodiments in which dopants (for example the rhenium component and/or the rhenium co-promoter) are deposited simultaneously with the deposition of a portion of silver, as opposed to deposition separate from the deposition of silver, a more favourable distribution of the dopant over the support will be achieved. Such embodiments are therefore preferred, as they will yield better catalysts. Silver may be divided over the various depositions, such that in each deposition a silver solution of the same silver concentration is employed. It is preferred, however, to employ in a later deposition a silver solution having a higher silver concentration than in a previous deposition.

The catalyst preferably comprises, in addition to silver and a high-selectivity dopant, if any, a component comprising a further element. Eligible further elements may be selected from the group of nitrogen, fluorine, alkali metals, alkaline earth metals, titanium, hafnium, zirconium, vanadium, thallium, thorium, tantalum, niobium, gallium and germanium and mixtures thereof. Preferably the alkali metals are selected from potassium, rubidium and cesium. Most preferably the alkali metal is potassium and/or cesium. Preferably the alkaline earth metals are selected from calcium and barium. Typically, the further element is present in the catalyst in a total quantity of from 0.01 to 500 mmole/kg, more typically from 0.05 to 100 mmole/kg, calculated as the element on the catalyst. Where possible, the further element may suitably be provided as an oxide or as an oxyanion, for example, as a sulfate, nitrate, nitrite, borate or molybdate, in salt or acid form. Salts of alkali metals or alkaline earth metals are suitable. The component comprising the further element may be employed in the invention in a quantity sufficient to provide a catalyst having a content of the further element as disclosed herein. The component comprising the further element may be deposited on the support prior to, together with or after the deposition of silver; and/or prior to, together with or after the deposition of the high-selectivity dopant, if any.

The content of alkali metal components of the catalyst generally influences the performance of the catalyst in the preparation of an olefin oxide from the olefin and oxygen. In one aspect, the performance may relate to the ability to operate the catalyst outside the conditions of a runaway reaction, that is total oxygen conversion and locally a very high catalyst temperature. In certain embodiments relating to catalysts which do not comprise a rhenium component or have a content of a rhenium component of less than 1.5 mmole/kg, in particular less than 1 mmole/kg, calculated as the quantity of rhenium relative to the weight of the catalyst, a cesium component may be applied in a quantity higher than the quantity that may be needed for optimal catalyst performance in terms of activity and selectivity (for example, 700 ppmw, instead of 500 ppmw, as the weight of cesium relative to the weight of the catalyst) with the effect that conditions of a runaway reaction are more easily avoided.

As used herein, the quantity of alkali metal present in the catalyst is deemed to be the quantity in so far as it can be extracted from the catalyst with de-ionized water at 100° C. The extraction method involves extracting a 10-gram sample of the catalyst three times by heating it in 20 ml portions of de-ionized water for 5 minutes at 100° C. and determining in the combined extracts the relevant metals by using a known method, for example atomic absorption spectroscopy.

As used herein, the quantity of alkaline earth metal present in the catalyst is deemed to be the quantity in so far as it can be extracted from the catalyst with 10% w nitric acid in de-ionized water at 100° C. The extraction method involves extracting a 10-gram sample of the catalyst by boiling it with a 100 ml portion of 10% w nitric acid for 30 minutes (1 atm., i.e. 101.3 kPa) and determining in the combined extracts the relevant metals by using a known method, for example atomic absorption spectroscopy. Reference is made to U.S. Pat. No. 5,801,259, which is incorporated herein by reference.

Although the present epoxidation process may be carried out in many ways, it is preferred to carry it out as a gas phase process, i.e. a process in which the feed is contacted in the gas phase with the catalyst which is present as a solid material, typically in a packed bed. Generally the process is carried out as a continuous process.

The olefin for use in the present epoxidation process may be any olefin, such as an aromatic olefin, for example styrene, or a di-olefin, whether conjugated or not, for example 1,9-decadiene or 1,3-butadiene. Typically, the olefin is a monoolefin, for example 2-butene or isobutene. Preferably, the olefin is a mono-α-olefin, for example 1-butene or propylene. The most preferred olefin is ethylene.

The olefin concentration in the feed may be selected within a wide range. Typically, the olefin concentration in the feed will be at most 80 mole-%, relative to the total feed. Preferably, it will be in the range of from 0.5 to 70 mole-%, in particular from 1 to 60 mole-%, on the same basis. As used herein, the feed is considered to be the composition which is contacted with the catalyst.

The present epoxidation process may be air-based or oxygen-based, see “Kirk-Othmer Encyclopedia of Chemical Technology”, 3^{rd} edition, Volume 9, 1980, pp. 445-447. In the air-based process air or air enriched with oxygen is employed as the source of the oxidizing agent while in the oxygen-based processes high-purity (at least 95 mole-%) oxygen is employed as the source of the oxidizing agent. Presently most epoxidation plants are oxygen-based and this is a preferred embodiment of the present invention.

The oxygen concentration in the feed may be selected within a wide range. However, in practice, oxygen is generally applied at a concentration which avoids the flammable regime. Typically, the concentration of oxygen applied will be within the range of from 1 to 15 mole-%, more typically from 2 to 12 mole-% of the total feed.

In order to remain outside the flammable regime, the concentration of oxygen in the feed may be lowered as the concentration of the olefin is increased. The actual safe operating ranges depend, along with the feed composition, also on the reaction conditions such as the reaction temperature and the pressure.

An organic halide may be present in the feed as a reaction modifier for increasing the selectivity, suppressing the undesirable oxidation of olefin or olefin oxide to carbon dioxide and water, relative to the desired formation of olefin oxide. Organic halides are in particular organic bromides, and more in particular organic chlorides. Preferred organic halides are chlorohydrocarbons or bromohydrocarbons. More preferably they are selected from the group of methyl chloride, ethyl chloride, ethylene dichloride, ethylene dibromide, vinyl chloride or a mixture thereof. Most preferred are ethyl chloride and ethylene dichloride.

The organic halides are generally effective as reaction modifier when used in low concentration in the feed, for example up to 0.01 mole-%, relative to the total feed. In particular when the olefin is ethylene, it is preferred that the organic halide is present in the feed at a concentration of at most 50×10⁻⁴ mole-%, in particular at most 20×10⁻⁴ mole-%, more in particular at most 15×10⁻⁴ mole-%, relative to the total feed, and preferably at least 0.2×10⁻⁴ mole-%, in particular at least 0.5×10⁻⁴ mole-%, more in particular at least 1×10⁻⁴ mole-%, relative to the total feed.

In addition to the olefin, oxygen and the organic halide, the feed may contain one or more optional components, for example carbon dioxide, inert gases and saturated hydrocarbons. Carbon dioxide is a by-product in the epoxidation process. However, carbon dioxide generally has an adverse effect on the catalyst activity. Typically, a concentration of carbon dioxide in the feed in excess of 25 mole-%, preferably in excess of 10 mole-%, relative to the total feed, is avoided. A concentration of carbon dioxide as low as 1 mole-% or lower, for example 0.5 mole-%, relative to the total feed, may be employed. Inert gases, for example nitrogen or argon, may be present in the feed in a concentration of from 30 to 90 mole-%, typically from 40 to 80 mole-%. Suitable saturated hydrocarbons are methane and ethane. If saturated hydrocarbons are present, they may be present in a quantity of up to 80 mole-%, relative to the total feed, in particular up to 75 mole-%. Frequently they are present in a quantity of at least 30 mole-%, more frequently at least 40 mole-%. Saturated hydrocarbons may be added to the feed in order to increase the oxygen flammability limit.

The epoxidation process may be carried out using reaction temperatures selected from a wide range. Preferably the reaction temperature is in the range of from 150 to 340° C., more preferably in the range of from 180 to 325° C.

In order to reduce the effects of deactivation of the catalyst, the reaction temperature may be increased gradually or in a plurality of steps, for example in steps of from 0.1 to 20 ° C., in particular 0.2 to 10° C., more in particular 0.5 to 5° C. The total increase in the reaction temperature may be in the range of from 10 to 140° C., more typically from 20 to 100° C. The reaction temperature may be increased typically from a level in the range of from 150 to 300° C., more typically from 200 to 280° C., when a fresh catalyst is used, to a level in the range of from 230 to 340° C., more typically from 240 to 325° C., when the catalyst has decreased in activity due to aging.

The epoxidation process is preferably carried out at a reactor inlet pressure in the range of from 1000 to 3500 kPa. “GHSV” or Gas Hourly Space Velocity is the unit volume of gas at normal temperature and pressure (0° C., 1 atm, i.e. 101.3 kPa) passing over one unit volume of packed catalyst per hour. Preferably, when the epoxidation process is as a gas phase process involving a packed catalyst bed, the GHSV is in the range of from 1500 to 10000 Nl/(l.h). Preferably, the process is carried out at a work rate in the range of from 0.5 to 10 kmole olefin oxide produced per m³ of catalyst per hour, in particular 0.7 to 8 kmole olefin oxide produced per m³ of catalyst per hour, for example 5 kmole olefin oxide produced per m³ of catalyst per hour.

The olefin oxide produced may be recovered from the reaction mixture by using methods known in the art, for example by absorbing the olefin oxide from a reactor outlet stream in water and optionally recovering the olefin oxide from the aqueous solution by distillation. At least a portion of the aqueous solution containing the olefin oxide may be applied in a subsequent process for converting the olefin oxide into a 1,2-diol, a 1,2-diol ether or an alkanolamine.

The olefin oxide produced in the epoxidation process may be converted into a 1,2-diol, into a 1,2-diol ether or into an alkanolamine.

The conversion into the 1,2-diol or the 1,2-diol ether may comprise, for example, reacting the olefin oxide with water, suitably using an acidic or a basic catalyst. For example, for making predominantly the 1,2-diol and less 1,2-diol ether, the olefin oxide may be reacted with a ten fold molar excess of water, in a liquid phase reaction in presence of an acid catalyst, e.g. 0.5-1.0% w sulfuric acid, based on the total reaction mixture, at 50-70 ° C. at 100 kPa absolute, or in a gas phase reaction at 130-240 ° C. and 2000-4000 kPa absolute, preferably in the absence of a catalyst. If the proportion of water is lowered the proportion of 1,2-diol ethers in the reaction mixture is increased. The 1,2-diol ethers thus produced may be a di-ether, tri-ether, tetra-ether or a subsequent ether. Alternative 1,2-diol ethers may be prepared by converting the olefin oxide with an alcohol, in particular a primary alcohol, such as methanol or ethanol, by replacing at least a portion of the water by the alcohol.

The conversion into the alkanolamine may comprise reacting the olefin oxide with an amine, such as ammonia, an alkyl amine or a dialkylamine. Anhydrous or aqueous ammonia may be used. Anhydrous ammonia is typically used to favor the production of monoalkanolamine. For methods applicable in the conversion of the olefin oxide into the alkanolamine, reference may be made to, for example U.S. Pat. No. 4,845,296, which is incorporated herein by reference.

The 1,2-diol and the 1,2-diol ether may be used in a large variety of industrial applications, for example in the fields of food, beverages, tobacco, cosmetics, thermoplastic polymers, curable resin systems, detergents, heat transfer systems, etc. The alkanolamine may be used, for example, in the treating (“sweetening”) of natural gas.

Unless specified otherwise, the organic compounds mentioned herein, for example the olefins, 1,2-diols, 1,2-diol ethers, alkanolamines and organic halides, have typically at most 40 carbon atoms, more typically at most 20 carbon atoms, in particular at most 10 carbon atoms, more in particular at most 6 carbon atoms. As defined herein, ranges for numbers of carbon atoms (i.e. carbon number) include the numbers specified for the limits of the ranges.

Having generally described the invention, a further understanding may be obtained by reference to the following examples, which are provided for purposes of illustration only and are not intended to be limiting unless otherwise specified.

### EXAMPLE 1

Preparation of Catalysts 1, 2 and 3

An impregnation solution was prepared by adding aqueous solutions comprising lithium hydroxide, cesium hydroxide, and water to samples of an silver-amine-oxalate stock solution. The amounts of lithium hydroxide and cesium hydroxide were 47 mmole/kg and 4 mmole/kg, respectively, relative to the weight of the support. The measured pH (20° C.) of the impregnation solution was 13.6. The silver-amine-oxalate stock solution was prepared as described in U.S. Pat. No. 4,766,105, which is incorporated herein by reference.

A sample of an α-alumina support having a surface area of 0.87 m²/g and a water absorption of 0.42 g/g was impregnated with the impregnation solution and dried, as follows. The support sample (approximately 30 g) was placed under a 25 mm Hg vacuum for 1 minute at ambient temperature. Approximately 50 g of the impregnating solution, prepared as indicated hereinbefore, was then introduced to submerse the support, and the vacuum was maintained at 25 mm Hg for an additional 3 minutes. The vacuum was then released and the excess impregnating solution was removed from the catalyst pre-cursor by centrifugation at 500 rpm for two minutes. The catalyst pre-cursor was then dried while being shaken at 250° C. for 5.5 minutes in a stream of air. The catalyst prepared contained 14.5% w silver, 40 mmole/kg lithium, and 3.4 mmole/kg cesium, relative to the weight of the catalyst (Catalyst 1, for comparison).

A second catalyst was prepared in the same way as Catalyst 1, except that 70 mmole of lithium hydroxide/kg support was used, instead of 47 mmole/kg. The measured pH (20° C.) of the impregnation solution was 14.6. The catalyst prepared contained 14.5% w silver, 60 mmole/kg lithium, and 3.4 mmole/kg cesium, relative to the weight of the catalyst (Catalyst 2, according to the invention).

A third catalyst was prepared in the same way as Catalyst 1, except that 70 mmole of lithium hydroxide/kg support was used, instead of 47 mmole/kg, and that the support was washed with water following the procedure outlined in U.S. Pat. No. 6,368,998, which is incorporated herein by reference. The measured pH (20 ° C.) of the impregnation solution was 14.6. The catalyst prepared contained 14.5% w silver, 60 mmole/kg lithium, and 3.4 mmole/kg cesium, relative to the weight of the catalyst (Catalyst 3, according to the invention).

Catalyst Testing

The catalysts prepared were tested in the production of ethylene oxide from ethylene and oxygen. To do this, 1.68 g of crushed catalyst was loaded into a stainless steel U-shaped tube. The tube was immersed in a molten metal bath (heat medium) and the ends were connected to a gas flow system. A gas or gas mixture passed through the catalyst bed, in a “once-through” operation. The weight of catalyst used and the inlet gas flow rate were adjusted to give a gas hourly space velocity of 6800 Nml of gas per ml catalyst per hour, as calculated for uncrushed catalyst. The inlet gas pressure was 1450 kPa.

The gas mixture contained 30% v ethylene, 8% v oxygen, 5% v carbon dioxide, 2.5 ppmv ethyl chloride, and nitrogen balance.

The reactor temperature was ramped up at a rate of 10° C. per hour to 225° C. and then the temperature was adjusted so as to achieve an ethylene oxide content of 1.5% v in the outlet gas stream. The ethyl chloride concentration in the gas mixture was adjusted between 2.5 and 5 ppmv so as to obtain an optimum selectivity at a constant ethylene oxide concentration in the outlet gas stream. The temperature was slowly increased to compensate for a decline in catalyst performance as a result of aging, i.e. such that a constant ethylene oxide content in the outlet gas stream was maintained.

The initial performance of the catalyst (i.e. after the catalyst had been on stream for at least 1-2 days) was measured and reported in Table I. A lower temperature is indicative of a higher activity of the catalyst. It can be seen that the Catalysts 2 and 3 (according to the invention) outperform comparative Catalyst 1.

TABLE I

| Catalyst | Selectivity (%) | Temperature (° C.) |
|---|---|---|
| 1 *) | 82.1 | 227 |
| 2 **) | 82.2 | 223 |
| 3 **) | 82.4 | 222 |

*) for comparison
**) according to the invention

The stability of the catalysts was evaluated as follows. Samples of the crushed catalysts (0.808 g) were each loaded in a 3.6 mm inside diameter stainless steel U-shaped tube. The tube was immersed in a molten metal bath (heat medium) and the ends were connected to a gas flow system. A gas or gas mixture passed through the catalyst bed, in a “once-through” operation. The weight of catalyst used and the inlet gas flow rate were adjusted to give a gas hourly space velocity of 30000 Nml of gas per ml catalyst per hour, as calculated for uncrushed catalyst. The inlet gas pressure was 1450 kPa.

The gas mixture contained 30% v ethylene, 8% v oxygen, 5% v carbon dioxide, 5.6 ppmv ethyl chloride, and nitrogen balance.

The reactor temperature was ramped up at a rate of 10° C. per hour to 245° C. and then the temperature was adjusted so as to achieve an oxygen conversion level of 25%.

After reaching the initial performance level of the catalysts, the temperature was slowly increased to compensate for a decline in catalyst performance as a result of aging, i.e. such that a constant oxygen conversion was maintained. There was a decline in catalyst performance in two stages. In the first stage the rate of decline in catalyst performance was substantially lower than in the second stage. In the first stage, virtually no catalyst selectivity decline was observed, while in the second stage, catalyst selectivity decline followed a substantially linear fashion, at a rate of about 1.56% per kton/m³ catalyst for all three catalysts. However, the starting point of the second stage, expressed in a cumulative ethylene oxide (EO) production, was significantly different for the three catalysts, as specified in Table II. Advantageously, the second stage decline started at a much higher cumulative EO production for inventive Catalysts 2 and 3, than for comparative Catalysts 1. Also, Catalyst 3 was significantly better than Catalyst 2.

TABLE II

| | | | | | Cumulative |
|---|---|---|---|---|---|
| | | | | | EO |
| | | | Performance at | | production |
| | Initial | | 1.3 kton/m³ | | at start |
| | Performance | | catalyst | | of second |
| Cata- | Selectivity | Temperature | Selectivity | Temperature | stage decline |
| lyst | (%) | (° C.) | (%) | (° C.) | (kton/m³) |
| 1*) | 80.4 | 257 | 78.4 | 268 | 0.08 |
| 2**) | 80.7 | 255 | 79.1 | 266 | 0.4 |
| 3**) | 80.9 | 247 | 80.9 | 256 | 1.6 |

*)for comparison
**)according to the invention

1. A process for preparing a catalyst comprising silver on a support, which process comprises
depositing silver on the support, and
depositing on the support a base having a pK_{b} of at most 3.5 when measured in water at 25 ° C., in a quantity of at least 50 mmole per kg of the support, simultaneously with or later than depositing silver on the support.

2. The process as claimed in claim 1, wherein the process comprises impregnating the support with a solution comprising a silver compound and a base having a pK_{b} of at most 3.5 when measured in water at 25° C. in a quantity of at least 50 mmole per kg of the support.

3. The process as claimed in claim 2, wherein the solution has a pH of at least 14 when measured at a temperature of 20° C.

4. The process as claimed in claim 1, wherein the base has a pK_{b} of at most 2, when measured in water at 25° C.

5. The process as claimed in claim 4, wherein the base has a pK_{b} of at most 1, when measured in water at 25° C.

6. The process as claimed in claim 1, wherein the base is a hydroxide or an alkoxide.

7. The process as claimed in claim 6, wherein the base is lithium hydroxide.

8. The process as claimed in claim 1, wherein the base is deposited in a quantity in the range of from 55 to 500 mmole/kg, relative to the weight of the support.

9. The process as claimed in claim 8, wherein the base is deposited in a quantity in the range of from 60 to 100 mmole/kg, relative to the weight of the support.

10. The process as claimed in claim 1, wherein silver is deposited on the support in a quantity in the range of from 100 to 400 g/kg, relative to the weight of the catalyst.

11. The process as claimed in claim 1, wherein, in addition to silver and the base, one or more high-selectivity dopants comprising one or more of rhenium, molybdenum, chromium and tungsten are deposited on the support.

12. The process as claimed in claim 11, wherein the high selectivity dopants are deposited in a quantity sufficient to provide a catalyst having a total content of high-selectivity dopant of from 0.01 to 500 mmole/kg, calculated as the element, that is the total of rhenium, molybdenum, chromium and/or tungsten, on the total catalyst.

13. The process as claimed in claim 11, wherein, in addition to silver and the base, a rhenium component and a rhenium co-promoter selected from components which comprise one or more of tungsten, chromium, molybdenum, sulfur, phosphorus and boron are deposited on the support.

14. The process as claimed in claim 13, wherein the rhenium component and the rhenium co-promoter are deposited in a quantity sufficient to provide a catalyst having a content of the rhenium component in the range of from 0.01 to 50 mmole/kg, calculated as the quantity of rhenium relative to the weight of the catalyst, and having a total content of the rhenium co-promoter in the range of from 0.01 to 50 mmole/kg, calculated as the element, that is the total of tungsten, chromium, molybdenum, sulfur, phosphorus and/or boron, relative to the weight of the catalyst.

15. The process as claimed in claim 14, wherein the rhenium component and the rhenium co-promoter are deposited in a quantity sufficient to provide a catalyst having a content of the rhenium component in the range of from 0.1 to 10 mmole/kg, calculated as the quantity of rhenium relative to the weight of the catalyst, and having a total content of the rhenium co-promoter in the range of from 0.1 to 10 mmole/kg, calculated as the element, that is the total of tungsten, chromium, molybdenum, sulfur, phosphorus and/or boron, relative to the weight of the catalyst.

16. A catalyst prepared in accordance with the process of claim 1.

17. A process for preparing an olefin oxide by reacting an olefin with oxygen in the presence of a catalyst, wherein the catalyst is prepared by a process which comprises
depositing silver on the support, and
depositing on the support a base having a pK_{b} of at most 3.5 when measured in water at 25° C., in a quantity of at least 50 mmole per kg of the support, simultaneously with or later than depositing silver on the support.

18. A process for producing a 1,2-alkanediol, a 1,2-alkanediol ether or a 1,2-alkanolamine comprising:
obtaining an olefin oxide by reacting an olefin with oxygen in the presence of a catalyst, wherein the catalyst is prepared by a process which comprises
depositing silver on the support, and
depositing on the support a base having a pK_{b} of at most 3.5 when measured in water at 25° C., in a quantity of at least 50 mmole per kg of the support, simultaneously with or later than depositing silver on the support; and
converting the olefin oxide into the 1,2-alkanediol, the 1,2-alkanediol ether, or the 1,2-alkanolamine.
